(19) United States
(12) Patent Application Publication
(10) Pub. No.: US 2004/0001794 A1
(43) Pub. Date: Jan. 1, 2004

(54) Compositions comprising odor neutralizing calcium silicate

(76) Inventors: Michael C. Withiam, Landenberg, PA (US); Sung-Tsuen Liu, Aberdeen, MD (US); Donald P. Conley, Conowingo, MD (US); Michael Simone, North East, MD (US)

Correspondence Address: D. Mitchell Goodrich, J.M. Huber Corporation, 333 Thornall Street, Edison, NJ, 08837-2220, US

(21) Appl. No.: 10/185,673

(22) Filed: Jun. 28, 2002

(51) Int. Cl. A61K 7/32; A61L 9/00; A61L 9/01

(52) U.S. Cl. 424/65

(57) ABSTRACT

Disclosed is a fluid personal care composition comprising calcium silicate and a vehicle, wherein the calcium silicate is capable of absorbing a malodorous compound. These fluid personal care compositions that provide effective, long-lasting suppression of the malodors associated with human perspiration. The personal care composition may be in the form of a solid stick deodorants, liquid roll-on deodorants, aerosol, and pump spray deodorants, semi-solid gel deodorants, soap bars, and deodorant lotions and creams

### BACKGROUND OF THE INVENTION

A broad array of topical personal care and personal hygiene products are available for application to human skin to counteract malodors associated with the human body, particularly those malodors resulting from and associated with perspiration. These products include deodorants, antiperspirants, foot and body powders, body sprays, and especially sports and athletic sprays and powders.

Conventional personal care and cosmetic products may be formulated to counteract and neutralize body malodors in a variety of ways. For instances, these malodors may be “masked” or concealed by placing a sufficient amount of perfume composition in the deodorant in order to hide or cover the malodor. Perfumes provide the additional benefit of imparting a desirable fragrance, such as a variety of different fresh, pastoral, or musk scents, to a cosmetic or personal care product. However, “masking” also has distinct limitations. Some malodors cannot be masked simply by adding perfumes, because they are highly volatile (and therefore diffuse quickly into the air) or because they are extremely potent. Indeed, in some cases it may be impossible to add sufficient amounts of perfume in order to sufficiently conceal the underlying malodor without also giving the personal care product an overly strong, perfumed odor.

Another way of counteracting body malodor is through the use of topical antimicrobials such as triclosan. Perspiration-associated body malodors are typically the result of interaction between microbes, perspiration and triglyceride secretions from the sebaceous glands, which combine to produce malodorous and pungent fatty acids. Thus, by controlling the microbe population on the skin's surface, the malodor can be eliminated or reduced in intensity.

However, the use of antimicrobial agents also presents certain problems. Overuse of antimicrobial agents is strongly discouraged because it can contribute to the development of disease-resistant microbes, and additionally the build-up of antimicrobial agents in the human body may have unknown side effects. Moreover, adding these antimicrobials to a typical deodorant composition, may cause the deodorant to irritate the skin.

Another approach that avoids the aforementioned problems while also reducing malodor involves the use of odor absorbers, such as activated charcoal and zeolites. These odor absorbing compounds function by absorbing odors and perspiration, and unlike the aforementioned treatment compounds they do not irritate the skin or impart an overly perfumed scent to the composition. However, charcoal and zeolite odor absorbers have the disadvantage that as they get wet (e.g., they come into contact with perspiration) they become ineffective at odor absorption. For similar reasons, these odor absorbers can also be difficult to formulate into compositions that contain even small quantities of water.

Given the forgoing there is a continuing need for cosmetic and personal care products that contain an ingredient to effectively suppress the malodors associated with human perspiration. Such a malodor-suppressing ingredient preferably has no other health or hygienic side effects, and can be easily formulated into a wide variety of cosmetic and personal care products.

### BRIEF SUMMARY OF THE INVENTION

The present invention includes a fluid personal care product comprising calcium silicate and a vehicle, wherein the calcium silicate is capable of absorbing a malodorous compound.

The invention also includes a method of inhibiting body odor by applying to the skin an effective amount of a personal care composition comprising calcium silicate capable of absorbing volatilized malodors.

### DETAILED DESCRIPTION OF THE INVENTION

All parts, percentages and ratios used herein are expressed by weight unless otherwise specified. All documents cited herein are incorporated by reference.

By “fluid personal care compositions” it is meant compositions that contain greater than 20% by weight of one or more ingredients acceptable for use in cosmetics that are liquid at temperatures less than 100° C., such as deodorants, anti-perspirants, athletic sprays, body sprays, hair conditioners, shampoo, skin conditioners, body washes, liquid bath soaps, facial cleansers, make-up removers, baby baths, hand soaps and the like.

The present invention includes topical fluid personal care compositions comprising an odor-absorbing, odor-neutralizing calcium silicate along with at least one acceptable vehicle (such as diluents or carriers) for the odor-absorbing calcium silicate, so as to facilitate the calcium silicate's distribution when the composition is applied to the skin. (Suitable vehicles, as well as other suitable personal care composition ingredients are discussed in greater detail, below). The silicates act as odor absorbents and neutralizers to absorb and neutralize body malodors, particularly body malodors associated with perspiration. By incorporating these calcium silicates, a wide variety of personal care compositions may be produced that provide effective, long-lasting absorption and neutralization of odors. This allows effective body malodor suppression without the overuse of perfumes or antimicrobial agents. In addition to these benefits, the calcium silicate also improves the “feel” of personal care compositions in which it is incorporated. Particularly, the personal care compositions have a smoother feel when applied and in contact with the skin.

That fluid personal care compositions incorporating calcium silicate are capable of providing effective odor neutralization and suppression would itself be surprising to a person of ordinary skill in the art. This is because the particulate calcium silicates are coated with several other ingredients, and thus would seem incapable of neutralizing and suppressing body malodors. However, by the present invention fluid personal care compositions have been formulated that fully incorporate calcium silicate particles without diminishing the ability of the calcium particles to absorb and neutralize odors.

While not wishing to be limited by theory, it is believed that the calcium silicates in the cosmetic compositions prepared according to the present invention absorb both malodors originating from human skin as well as absorb the fatty acids found on the skin. Thus, these calcium silicates are believed to offer two measures to neutralize body malodors: they not only absorb the malodors themselves, but they also reduce the quantities of fatty acids that are part of the cause of the malodors. The high oil absorption capacity of the calcium silicates encourage the movement of the malodorous compounds into the intraparticle pores and interstices that are formed within the calcium silicates.

These excellent absorption and neutralization properties are themselves believed to result from the high surface area and oil absorption properties of the calcium silicates. The high surface area of the calcium silicate particles allows for the volatilized malodors and fatty acids to be easily adsorbed onto the surfaces of the silicate particles, while the high oil absorption capacity of the calcium silicates encourages the movement of volatilized malodors and fatty acids from the particle surface and into the interior of the calcium silicate particles.

Thus, the present fluid personal care compositions contain synthetic amorphous calcium silicates that absorb volatilized malodors and fatty acids and thereby neutralize the malodors associated with human perspiration. Preferably the oil absorption of these silicates is greater than 200 ml/100 g, preferably between 400 ml/100 g and 600 ml/100 g, most preferably around 475 ml/100 g. The surface area (BET) is preferably around 150 m²/g to 600 m²/g, preferably between 300 m²/g and 600 m²/g, actually between 310 m²/g and 350 m²/g. The particle size is preferably less than 20 microns.

These calcium silicates are most typically prepared by the reaction of a reactive silica with an alkaline earth metal reactant, preferably an alkaline earth metal oxide or hydroxide, and a source of aluminum such as sodium aluminate or alumina. Because the final properties of the silicate are dependent on the reactivity of the silica, the silica source is preferred to be the reaction product of a soluble silicate, such as, but not limited to sodium silicate, and a mineral acid, such as sulfuric acid. Suitable synthetic amorphous alkaline earth metal silicates are manufactured by the J. M. Huber Corporation and are sold in different grades under the trademark Hubersorb®. Methods and techniques for preparing these silicates are discussed in greater detail in U.S. Pat. No. 4,557,916. Other suitable amorphous silicates are available from J. M. Huber Corporation such as sodium aluminosilicate sold under the trademark Zeolex® and sodium magnesium aluminosilicate sold under the trademark Hydrex®.

Physical characteristics of calcium silicate used in the present invention, as well as other absorbent materials are given below in Table A.

TABLE A
Physical Properties of Absorbents

| | | Specific | Oil Absorption | Loose |
|---|---|---|---|---|
| | Median | surface | Linseed oil rub- | bulk |
| | particle size, | area, BET, | out method, | density, |
| Absorbent | μm | m2/g | cc/100 g | g/ml |
| PolyGloss ® 90 | 0.4 | 22 | 42 | 2.6 |
| Zeofree ® 80 | 14 | 140 | 200 | 0.14 |
| Zeothix ® 265 | 4 | 250 | 220 | 0.10 |
| Cab-O-Sil ® M5 | 9 | 207 | 189 | 0.04 |
| Hubersorb ® 600 | 5 | 320 | 475 | 0.13 |

Polygloss® 90, a kaolin clay, Zeofree® 80 and Zeothix® 265, synthetic amorphous precipitated silicas, and Hubersorb®, 600 a calcium silicate, are all available from the J. M. Huber Corporation. Cab-O-Sil® M5 synthetic amorphous fumed silica is available from the Cabot Corporation, Bellrica, Mass. The odor absorption and neutralization properties of some of these materials are discussed in greater detail in the Examples, below.

Where mentioned in this application, the surface area was determined by the BET nitrogen absorption method of Brunaur et al., as reported in the J. Am. Chem. Soc. 60, 309 (1938). Bulk density is determined by weighing 100.0 grams product into a 250-mL graduated cylinder and recording the volume occupied.

Particle size is determined using a Model LA-910 laser light scattering instrument available from Horiba Instruments, Boothwyn, Pa. A laser beam is projected through a transparent cell, which contains a stream of moving particles suspended in a liquid. Light rays, which strike the particles, are scattered through angles which are inversely proportional to their sizes. The photodetector array measures the quantity of light at several predetermined angles. Electrical signals proportional to the measured light flux values are then processed by a microcomputer system to form a multi-channel histogram of the particle size distribution.

The oil absorption shown in table A (and throughout this application) is measured with the rubout method. In this test, oil is mixed with a silicate and rubbed with a spatula on a smooth surface until a stiff putty-like paste is formed. By measuring the quantity of oil required to have a paste mixture, which will curl when spread out, one can calculate the oil absorption value of the silicate, the value which represents the volume of oil required per unit weight of silicate to completely saturate the silicate absorptive capacity. Calculation of the oil absorption value was done according to equation (I):

$$\begin{matrix}Oil absorption = \frac{ml oil absorbed}{weight of silicate , grams} \times 100 \\ = ml oil \text{/} 100 gram silicate\end{matrix} \qquad ( I )$$

Fluid personal care compositions prepared according to the present invention comprise about 0.5 wt % to about 20 wt %, preferably about 1 wt % to about 10 wt % of the odor neutralizing calcium silicate. In addition to the calcium silicate, the present fluid personal care compositions will also comprise one or more dermatologically acceptable cosmetic ingredients.

Dermatologically acceptable cosmetic ingredients include first and most importantly a diluent or carrier. The vehicle, diluent or carrier may be selected from a wide range of ingredients. The vehicle may comprise water and/or a water-miscible or dispersible organic liquid or liquids and alternatively or additionally a water-immiscible liquid or liquids and waxes. The cosmetically acceptable vehicle will preferably form from 80% to 99% by weight of the composition, and can, in the absence of other cosmetic adjuncts, form the balance of the composition. The vehicle may be aqueous, non-aqueous or a combination of both, such as an emulsion. In a combination vehicle, an oil or oily material may be present, together with one or more emulsifiers to provide either a water-in-oil emulsion or an oil-in-water emulsion, depending largely on the average hydrophilic-lipophilic balance (HLB) of the emulsifiers employed. This also includes multiple emulsions: water-in-oil-in-water or an oil-in-water-in-oil emulsions.

In the case where the composition contains a combination of aqueous and non-aqueous vehicle components, the aqueous phase can be from about 90 wt. % to about 10 wt. % of the vehicle, as can the non-aqueous phase. In an embodiment of the invention where the vehicle is aqueous or is comprised of a mixture of aqueous and non-aqueous components, preferably the vehicle is at least 80 wt % water, by weight of the vehicle. Preferably, water comprises at least 85 wt % of the inventive composition, and most preferably from 90 to 95 wt % of the composition.

In an embodiment of the invention where the vehicle is comprised of non-aqueous components, the dermatologically acceptable non-aqueous cosmetic ingredients in the vehicle will usually form from 80% to 99.9% by weight of the composition, and may, in the absence of other cosmetic adjuncts, form the balance of the composition.

Examples of suitable non-aqueous carriers may include alcohols, polyalkoxylated glycols (such as propylene glycol), volatile and nonvolatile liquid silicone carriers (such as cyclicsilicone polymers), hydrocarbon and mineral oils and branched chain hydrocarbons. Specific, non-limiting examples of organic liquids suitable for use include octyldodecanol, butyl stearate, diisopropyl malate, and combinations thereof. Also suitable for use are acrylic acid-based polymers.

It is desirable that the odor absorbing ingredient in the inventive compositions remains substantially localized in the region of the body to which it has been topically applied. In order to assist this to happen and also to enable alternative dispensers for the composition to be employed, the vehicle may be thickened or structured, for example by introducing one or more materials for that purpose. Thickened or structured compositions commonly adopt the form of firm sticks, soft solids and creams. In such circumstances, the materials are often referred to as structurants or gellants and may sometimes alternatively be called thickeners, depending on the final form of the composition. The vehicle may be further diluted with a volatile propellant and used as an aerosol; may be mixed with an additional liquid and/or other ingredients and used, for example, as a roll-on or squeeze-spray product; or mixed with one or more thickeners and/or structurants and used, for example, as a gel, soft solid, or solid stick product.

Exemplary thickeners are cross-linked polyacrylate materials available under the trademark Carbopol from the B. F. Goodrich Company. Gums may be employed such as xanthan, carrageenan, gelatin, karaya, pectin and locust beans gum. Under certain circumstances, the thickening function may be accomplished by a material also serving as a carrier or emollient vehicle. For instance, silicone gums in excess of 10 centistokes and esters such as glycerol stearate have such dual functionality. A thickener will usually be present in amounts anywhere from 0.1 to 20% by weight, preferably from about 0.5% to 10% by weight of the composition.

Other dermatologically acceptable cosmetic ingredients include rheology affecting agents such as solidifying agents and gellants. The solidifying agents act to provide solidity to a personal care composition so that they are in solid (or semi-solid) form at room temperature. Suitable solidifying agents include especially high melting point waxes (melting points between 65° C.-110° C.) which include hydrogenated castor oil, paraffin, synthetic wax, ceresin, beeswax, and other such waxes. Also acceptable are low melting point waxes (melting points between 37° C. -65° C.), which include fatty alcohols, fatty acids, fatty acids esters, fatty acid amides, and the like.

Gellants are used in the case of solid stick compositions, to give the stick an appropriate consistency and provide an appropriate gel matrix and product hardness at the completion of processing. The gelling agents will vary depending on the particular form of the personal care composition and whether the personal care composition is aqueous or nonaqueous. Suitable gellants include esters and amides of fatty acid or hydroxy fatty acid gallants, fatty acid gellants, salts of fatty acids, esters and amides of fatty acid or hydroxy fatty acid gellants, cholesterolic materials, lanolinolic materials, fatty alcohols, triglycerides, and other suitable solid, non-polymeric gellants. Preferred gellants (for both aqueous and nonaqueous compositions) include fatty alcohols, most preferably stearyl alcohol. Amounts of these gellant components may range anywhere from 0.001% up to 20% by weight of the composition.

The inventive compositions may contain any of a number of desired “active” ingredients, including drug substances such as anti-inflammatory agents, topical anesthetics, antimycotics, etc.; skin protectants or conditioners; humectants; and the like, depending on the intended uses for the formulations.

The fluid personal care products prepared according to the present invention may also include other optional components. The CTFA Cosmetic Ingredient Handbook, Eighth Edition, 2000, which is incorporated by reference herein in its entirety, describes a wide variety of cosmetic and pharmaceutical ingredients commonly used in skin care compositions, and which are suitable for use in the compositions of the present invention. These optional components include pH buffering agents, additional malodor control agents, fragrance materials, dyes, and pigments, preservatives, skin aids (e.g., aloe), cosmetic astringents, liquid or solid emollients, emulsifiers, film formers, propellants, skin-conditioning agents, such as humectants, skin protectants, solvents, solubilizing agents, suspending agents, surfactants, waterproofing agents, viscosity increasing agents (aqueous and nonaqueous), waxes, wetting agents, and other optional components. Amounts of these adjunct components may range anywhere form 0.001% up to 20% by weight of the composition.

The products themselves may be formulated to be in a variety of forms, such as solid and semi-solid stick deodorants (such as emulsion sticks or suspensoid sticks), roll-on deodorants, and deodorant aerosol and pump-sprays, and even soap bars.

The fluid personal care compositions of the present invention may be prepared by any known or otherwise effective technique suitable for providing a fluid personal care composition having the essential materials described herein. Techniques for forming such compositions are very well known in the art. The present invention is not dependent upon any particular formulation technique, it being recognized that the choice of specific formulation components may well make necessary some specific formulation procedure.

Methods for preparing the fluid personal care compositions of the present invention include conventional formulation and mixing techniques. Many variations of formulating the compositions of the present invention exist, and all are considered within the scope of the present invention. Suitable methods include combining the calcium silicate odor absorbing/neutralizing agent with part or all of the liquid vehicle. A liquid may be entirely absorbed into the calcium silicate, and if so, additional liquid or liquids and other materials are added until the calcium silicate is evenly dispersed. A thickener or gellant is added and the composition is mixed and may be heated, if required for homogenous incorporation. Adjunct and/or additional materials may be added at this point, and the batch may be allowed to cool, if necessary. The thickened or gelled composition is allowed to viscosify or solidify in a suitable container or dispenser.

The invention will now be described in more detail with respect to the following, specific, non-limiting examples.

### EXAMPLE 1

Trans-3-methyl-2-hexenoic acid is the principal active component associated with armpit (axilla) odor, however it was not available commercially. Isovaleric (3-methylbutanoic), 2-pentenoic and 2-hexenoic were chosen as the test substances, since they are closely chemically related trans-3-methyl-2-hexenoic acid. Isovaleric acid has been used in similar research.

Isovaleric acid, 2-pentenoic acid and 2-hexenoic acid are all associated with and contribute to foot and body perspirative malodors. Commercial samples of these malodorous materials were used as model compounds to assess the ability of cosmetic compositions prepared according to the present invention, comprising synthetic calcium silicate materials to remove the odors associated with these malodorous materials. Several other common materials known in the prior art to suppress malodors, such as talc, baking soda, and precipitated silica, were also included for comparative purposes.

In this example, first an aqueous solution comprising a concentration of 2000 μg /ml of isovaleric, 2000 μg /ml of 2-pentenoic and 2000 μg /ml of 2-hexenoic acids was prepared as an odor standard solution. Then, test specimens were prepared by adding 2 grams of an odor absorbing/neutralizing test compound to 50 ml of the odor standard solution in a glass container. These mixtures were capped, shaken, and allowed to sit for about a week. Then 5 ml aliquots of these mixtures were then transferred to 22-ml headspace autosampler vials, and 2 g of NaCl added. The resulting mixtures are then capped, shaken by hand and then analyzed using GCMS (“Gas Chromatography Mass Spectrometry”) headspace analysis to determine the detectable quantity of non-absorbed odor causing substance.

The GCMS analysis took place on a system consisting of a gas chromatograph (GC) directly attached to a mass selective detector (MS). Each gaseous solute exiting the GC is ionized in an electron beam. The ions formed by a specific solute will depend on the nature of the bonds in the molecule, and both ionized molecules and ion fragments of the molecule are possible. The ions are then directed down a separator, which isolates and counts the ions according to mass. The sequence and relative intensity of the mass peaks give information about the chemical identity of the solute. The absolute intensity of the peaks provides information about the amount of substance present: the greater the amount of peak area, the greater the amount of substance that is present.

A Hewlett Packard GCMS system used for the analyses consisted of a HP 7694 headspace auto-sampler, HP 5890 Gas Chromatograph and HP 5972 Mass Selector Detector. The GC was outfitted with a Restek RTX 624 Volatiles column (30 m length, 0.25 mm id., 1.4 μm film thickness.) The GCMS system was set to the following operating conditions:

TABLE I
GCMS Operating Conditions

| | |
|---|---|
| In the Headspace: | |
| Oven temp. | 80° C. |
| Transfer/loop | 200° C. |
| Equilibration time | 15 min. |
| Loop fill time | 0.02 min. |
| Loop pressurization time | 0.13 min |
| Injection time | 0.20 min. |
| Sample loop | 1 ml |
| For the GCMS: | |
| Temp profile: | 10 min @ 50° C. |
| | 20 min ramp to 250° C. |
| | 10 min @ 250° C. |
| Injection temp: | 220° C. |
| Detector temp: | 280° C. |

The results of the GCMS test are as follows:

TABLE II
Measured Peak Area of Malodorous Substances

| | Isovaleric acid | 2-Pentenoic acid | 2-Hexenoic acid |
|---|---|---|---|
| | Peak area | Peak area | Peak area |
| Samples | (000,000) | (000,000) | (000,000) |
| Odor standard | 15 | 3.1 | 5.7 |
| solution | | | |
| Corn Starch | 14 | 2 | 2 |
| Talc | 8 | 1 | 1 |
| silica A | 14 | 2 | 1 |
| silica B | 18 | 3 | 5 |
| clay | 9 | 1 | 2 |
| Baking Soda | nd | nd | nd |
| calcium silicate | nd | nd | nd |

In table II above, and throughout the application, raw peak area is dimensionless. Where used, “nd” indicates “none detected”, i.e., that the odor was below detectable levels. Samples were prepared at identical concentrations and analyzed under identical conditions giving a comparative analysis. Silica A is Zeofree® 80 silica, Silica B is Zeosyl® TG80 silica, the clay is Polygloss® 90 clay, and the calcium silicate is Hubersorb®600, all of which are available from the J. M. Huber Corporation of Edison, N.J.

As can be seen in Table II, synthetic calcium silicate and baking soda (sodium bicarbonate) were highly effective at reducing the concentration of the volatile isovaleric acid, 2-pentenoic acid, and 2-hexenoic acid (as indicated by the GC Peak area), reducing the amounts of these malodorous organic acids to non-detectable levels.

### EXAMPLE 2

In this example, test specimens were prepared by adding 10 μl of neat isovaleric acid and 10 μl of neat 2-pentenoic acid to 0.5 g of each of the odor absorbing/neutralizing sample test compounds listed in Table III, in a sample vial, which was then sealed with a crimp cap. The dry mixture was shaken by hand and analyzed immediately. These specimens (being a mixture of a sample of odor absorbing/neutralizing substance and malodorous acids) were analyzed by GCMS headspace analysis as described above. The odor standard solution consisted of 10 μl of neat isovaleric acid and 10 μl of neat 2-pentenoic acid sealed in a crimp cap vial, without an odor absorbing/neutralizing substance. The results are listed below in Table III.

TABLE III
Peak area by GCMS headspace analysis

| | Isovaleric acid | 2-Pentenoic acid |
|---|---|---|
| Sample | Peak area (000,000) | Peak area (000,000) |
| Odor standard solution | 61 | 17 |
| Talc | 53 | 13 |
| Corn Starch | 35 | 3 |
| Baking Soda | 0.9 | nd |
| Clay | 26 | 6 |
| Silica C | 12 | 2 |
| Silica D | 28 | 6 |
| calcium silicate | nd | nd |

Silica C is Cab-O-Sil® M5 from the Cabot Corporation, Bellrica, Mass. Silica D is Zeosyl® TG80 silica, the clay is the Polygloss® 90 clay, and the calcium silicate is Hubersorb® 600, all of which are available from the J. M. Huber Corporation.

As can be seen in Table III, synthetic calcium silicate resulted in essentially complete removal of the odoriferous substances isovaleric acid and 2-Pentenoic acid. Baking soda was also effective at removing malodors, but not as effective as calcium silicate.

### EXAMPLE 3

It was noted above that calcium silicate is more effective even than baking soda, a well known-prior art odor absorber and neutralizer. To provide further confirmation of calcium silicate's improved performance, as well as some analytical measurement of the degree of improvement, calcium silicate and baking soda were tested side-by-side.

To do this, isovaleric acid was added, incrementally to separate 0.5 g samples of the dry soda and calcium silicate powders. The calcium silicate showed no release of isovaleric acid at a 500 μl addition, while baking soda showed consistent release of isovaleric acid at a 50 μl addition. The sample specimens were tested using GCMS headspace analysis to determine the release or saturation point for each material. The results are set forth in Table IV, below.

TABLE IV

| | Isovaleric Acid |
|---|---|
| Sample | Capacity μl/g |
| Synthetic | 1000 |
| calcium silicate | |
| Baking Soda | <100 |

As can be seen in Table IV, the capacity of calcium silicate to absorb isovaleric acid was ten times greater than that of baking soda. This confirms that the odor absorbing capacity of calcium silicate is far greater than the capacity of baking soda.

### EXAMPLE 4

The calcium silicate materials of the present invention are useful not only directly in powder form, but they may also be incorporated into an aqueous polymer matrix, such as a carbomer gel system. In the present example, the carbomer gel that was used was Cabopol Resin EDT2020 (a crosslinked, acrylic acid-based polymer), available from Noveon, Inc., Cleveland, Ohio. Carbomer gel composition prepared accorded to the present invention included 5 wt % calcium silicate. For comparative purposes, a carbomer gel composition was also prepared that contained no odor neutralizer or absorber of any kind, and an additional carbomer gel control composition was also prepared that included 5 wt % talc, a prior art cosmetic ingredient. The carbomer gels were prepared by mixing the Carbopol resin, water and a odor absorbing/neutralizing compound. Thereafter, sodium hydroxide was added to initiate gel formation. The specific formulations for the carbomer gel compositions are set forth in Table V, below.

TABLE V
Carbomer Solid Gel Compositions

| | Carbomer | Carbomer Solid | Carbomer Solid gel |
|---|---|---|---|
| | Solid | gel (with 5 | (with 5 wt % |
| | gel | wt % Talc) | Calcium silicate) |
| Carbopol Resin | 0.5 | 0.5 | 0.5 |
| EDT2020, g | | | |
| Sodium hydroxide, g | 0.02 | 0.02 | 0.02 |
| Water, g | 99.48 | 94.48 | 94.48 |
| Talc, g | 0 | 5 | 0 |
| Calcium silicate, g | 0 | 0 | 5 |

The ability of these three compositions to absorb isovaleric acid was then tested as described above in Example 1. In the test, the ratio of the amount of isovaleric acid to the amount of calcium silicate (or talc) was 1 l of isovaleric acid to 1 mg of calcium silicate (or talc). Specifically, 5 g of each test formulation was weighed into a specimen vial, a crimp cap applied to the vial and then 250 μl isovaleric acid injected into the vial through the crimp cap septum, while the vial was being agitated on a vortex mixer (VM3000 mini-vortexer). Note that a 5 g specimen contained 250 mg calcium silicate (or talc). Mixing continued for about 30 seconds, thereafter the test specimens were immediately analyzed. As before, the isovaleric acid standard was 10 μl isovaleric acid sealed in a crimp-capped vial. The results of the test are set forth in Table VI, below.

TABLE VI
Carbomer Solid Gel Absorption of Isovaleric acid

| | GC Peak Area |
|---|---|
| | (000,000) |
| Isovaleric acid standard | 220 |
| Carbomer solid Gel Control | 131 |
| Carbomer solid Gel | 142 |
| (with 5 wt % talc) | |
| Carbomer solid Gel with | 29 |
| (with 5 wt % calcium silicate) | |

As can be seen in Table VI, the Carbomer solid gel containing the calcium silicate odor neutralizer prepared according to the present invention was considerably more effective at reducing the concentration of volatile isovaleric acid (as indicated by the GC Peak Area) when compared to the carbomer solid gel containing talc or to a carbomer solid gel control containing no odor neutralizer at all.

### EXAMPLE 5

Aqueous polymer semi-solid compositions were prepared containing odor absorbing calcium silicate as prepared according to the present invention. A comparative aqueous polymer semi-solid control composition was also prepared that contained no odor absorber or neutralizer. The compositions were prepared by mixing the CMC, Kelzan resin and water with a Lightnin mixer until a clear gel was formed. Thereafter, the calcium silicate was added and again mixed with the Lightnin mixer. The specific formulations are set forth in Table VII, below.

TABLE VII

| | Semi-solid | Semi-solid 5% |
|---|---|---|
| Formulation | Control | Calcium Silicate |
| Kelzan M Resin, g | 0.31 | 0.29 |
| CMC 7MF, g | 0.94 | 0.89 |
| Calcium silicate, g | 0 | 5 |
| Water, g | 98.8 | 93.9 |

Kelzan M Resin is xanthan gum available from CP Kelco of Chicago, Ill. CMC 7MF is carboxymethyl cellulose available form Aqualon Corporation of Wilmington, Del. The calcium silicate is Hubersorb 600.

The ability of these compositions to absorb isovaleric acid was then tested. In the test, isovaleric acid was added at a ratio of 1 l isovaleric acid to 1 mg calcium silicate based on the weight of calcium silicate in the formula. Test specimens and the isovaleric acid standard were prepared as described above. The results of the test are set forth in Table VIII, below.

TABLE VIII
Semi-solid Aqueous Polymer Absorption of Isovaleric acid

| | | Calcium | |
|---|---|---|---|
| | Isovaleric Acid | silicate | GC Peak Area |
| Formulation | (μl) | (mg) | (000,000) |
| Isovaleric acid | 10 | 0 | 180 |
| standard | | | |
| Semi-solid control | 250 | 0 | 107 |
| Semi-solid with 5.0 | 250 | 250 | 60 |
| wt % calcium silicate | | | |

As can be seen in Table VIII, the semi-solid aqueous polymer formulation containing the calcium silicate odor neutralizer prepared according to the present invention was considerably more effective at reducing the concentration of volatile isovaleric acid (as indicated by the GC Peak Area) when compared to the semi-solid aqueous polymer formulation containing no odor neutralizer at all.

### EXAMPLE 6

Many solid stick deodorant and antiperspirant products commercially available are based on cyclicsilicone (cyclopentasiloxane) polymers. Formulations were prepared that are representative of cyclicsilicone polymer sticks and incorporate calcium silicate as prepared according to the present invention. Specifically, the amounts of the product ingredients listed in Table IX below were added to a 250-ml Wolfe bottle. In one of the formulations, no odor absorbing fillers were used; in another formulation two prior art odor absorbing fillers, talc and aluminum zirconium tetrachlorohydrex (“AZT”), were used; while in a third formulation, 5 wt % of calcium silicate (Hubersorb® 600) was added. The aforementioned components were mixed in a Wolfe bottle, to which a water condenser is attached, and then heated to 80-85° C. with stirring on a magnetic hot/stir plate. After 15 minutes at this temperature, the heat is turned off and the mixture is allowed to cool to about 50-54° C., then transferred to a suitable container, such as a plastic bottle, for future testing. The mixture is still fluid at about 50° C., but solidifies in-situ in the container as it is cooled to room temperature.

TABLE IX
Cyclicsilicone Solid Stick Formulations

| | | Cyclicsilicone | |
|---|---|---|---|
| | Cyclicsilicone | 5% Calcium | Cyclicsilicone |
| Formulation | Solid Control | silicate | AZT/Talc |
| Cyclomethicone | 48.8 | 48.8 | 48.8 |
| (SF-1202), g | | | |
| Stearyl Alcohol | 15.0 | 15.0 | 15.0 |
| (Lanette 18 DEO), g | | | |
| Hydrogenated Castor | 2.0 | 2.0 | 2.0 |
| Oil (Castor Wax | | | |
| MP-80), g | | | |
| Tricontanyl PVP | 0.5 | 0.5 | 0.5 |
| (GANEX ® WP-660), | | | |
| g | | | |
| Phenyltrimethicone | 2.0 | 2.0 | 2.0 |
| (DC-556), g | | | |
| Talc, g | 0 | 0 | 5.85 |
| AZT, g | 0 | 0 | 25.45 |
| Calcium silicate, g | 0 | 3.59 | 0 |

The Cyclomethicone is SF-1202 available from GE Silicones Waterford, N.Y. Stearyl alcohol is Lanette 18 DEO available from Cognis Corp. Cincinnati, Ohio. The Hydrogenated Castor Oil is Castor Wax MP-80 available from Frank B. Ross Co. Jersey City, N.J. Tricontanyl PVP is GANEX® WP-660 available from Cognis Corp., Cincinnati, Ohio. Phenyltrimethicone is DC-556 available from Dow Corning, Taylor, Mich. The AZT is AGZ-370 available from Summit Research Labs, Huguenot, N.Y.

The ability of these cyclicsilicone (cyclic polysiloxane) solid stick formulation compositions to absorb malodorous isovaleric acid was then tested. Specifically, 5 g of each of the formulations described above in Table IX was separately placed into a headspace vial and sealed with a crimp-cap. The amount of isovaleric acid indicated in Table X below was injected through the cap septum while the vial was being agitated with a vortexer. The specimens were analyzed utilizing GCMS, under conditions described earlier. The results are shown in Table X, below.

TABLE X
Absorption of Isovaleric Acid by Cyclicsilicone Solid Stick
Formulations

| Formulation | GCC Peak Area (000,000) | | |
|---|---|---|---|
| μl isovaleric acid | 500 | 750 | 1000 |
| Cyclicsilicone Solid Control | 0 | 18 | 33 |
| Cyclicsilicone with | 4 | 6 | 11 |
| 5 wt % calcium silicate | | | |
| Cyclicsilicone with AZT and | 23 | 50 | 76 |
| Talc | | | |

As can be seen in Table X, the cyclicsilicone solid stick formulation containing the calcium silicate odor neutralizer prepared according to the present invention was extremely effective in reducing the concentration of volatile isovaleric acid (as indicated by the GC Peak Area), and significantly better than the prior art odor absorbers, AZT and talc.

### EXAMPLE 7

Commercial Mennen Speed Stick Ultimate® Alpine Fresh is a well-known commercial deodorant and antiperspirant stick containing AZT as an active ingredient, and is available from Colgate-Palmolive, New York, N.Y. This sample was compared to the cyclicsilicone solid stick, containing 5 wt. % calcium silicate, as described in Example 6 and to a Speed Stick Ultimate solid stick that was modified to contain 5 wt. % calcium silicate. The commercial solid Speed Stick Ultimate was heated at 80° C. in a water bath to melt the solid. To the liquefied solid, 5% wt/wt Hubersorb 600 calcium silicate was added to effect a homogenous mixture as described in Table XI.

TABLE XI
Commercial Deodorant Solid Stick

| | Mennen | Cyclicsilicone | Mennen with |
|---|---|---|---|
| | Commercial | with 5% Calcium | 5% Calcium |
| Formulation | Solid | silicate | silicate |
| Mennen Speed Stick | 100 | 0 | 95 |
| Solid, g | | | |
| Cyclicsilicone base form- | 0 | 95 | 0 |
| ula from Ex. 6, g | | | |
| Calcium silicate, g | 0 | 5 | 5 |

The ability of these compositions to absorb isovaleric acid was then tested. Test specimens were prepared by placing 2.5 g of each formulation separately into headspace vials and then sealing the vials with crimp caps. Through the cap septum, 250 μl of isovaleric acid was injected while the mixture was agitated with a vortex mixer. The specimens were then analyzed by GCMS under the conditions described earlier. GCMS determinations occurred on 2 different days, which explains the different values for the Mennen solid stick. The results of these tests are set forth in Table XII, below.

TABLE XII
Absorption of Isovaleric Acid by Cyclicsilicone Solid Stick
Formulations

| | GCC Peak Area | GCC Peak Area |
|---|---|---|
| Formulation | (000,000) | (000,000) |
| Cyclicsilicone with | 4.6 | — |
| 5 wt % calcium silicate | | |
| Mennen Solid Stick | 32 | 40 |
| Mennen Solid Stick with | — | 24 |
| 5 wt % calcium silicate | | |

As can be seen in Table XII, the solid stick formulation containing the calcium silicate as prepared according to the present invention was extremely effective at reducing the concentration of malodorous isovaleric acid (as indicated by the GC Peak Area). Additionally the addition of calcium silicate to the commercial solid further enhanced its ability to reduce the concentration of malodorous isovaleric acid by 40%.

### EXAMPLE 8

To evaluate the odor absorbing performance and efficacy of calcium silicate incorporated in a solid stick deodorant product as prepared according to the present invention, a solid stick deodorant formulation containing calcium silicate and similar to that of commercial stick deodorants was produced and tested on human subjects under conditions of actual use. Various cosmetic ingredients, as listed in Table XIII, below, were mixed in a Wolfe bottle, to which a water condenser is attached, and then heated to 80-85° C. with stirring on a magnetic hot/stir plate. Upon complete addition of all the other ingredients, the calcium silicate (in the amounts shown below) or triclosan (premixed with stearyl alcohol) was stirred in and the mixture allowed to cool to 53° C. before filling appropriate containers for clinical testing.

TABLE XIII
Cyclicsilicone Solid Stick Deodorant Formulation

| | Cyclicsilicone | Cyclicsilicone | Cyclicsilicone |
|---|---|---|---|
| | with 3% cal- | with 6% cal- | with 0.3% |
| | cium silicate | cium silicate | triclosan |
| Formulation | Test Article A | Test Article B | Test Article C |
| Cyclomethicone | 714.5 | 714.5 | 714.5 |
| (SF-1202) | | | |
| Stearyl Alcohol | 219.6 | 219.6 | 219.6 |
| (Lanette 18 DEO) | | | |
| Triclosan | 0 | 0 | 3.0 |
| Hydrogenated Castor | 29.3 | 29.3 | 29.3 |
| Oil (Castor Wax | | | |
| MP-80) | | | |
| Tricontanyl PVP | 7.3 | 7.3 | 7.3 |
| (GANEX ® WP-660) | | | |
| Phenyltrimethicone | 29.3 | 29.3 | 29.3 |
| (DC-556) | | | |
| Calcium silicate | 30 | 60 | 0 |

Clinical testing of approximately 40 subjects was made to compare the performance of the solid deodorant compositions of Table XIII containing calcium silicate at two different concentrations with identical formulations containing triclosan, but absent calcium silicate. The clinical test was conducted in compliance with applicable Good Clinical Practice Regulations in accordance with Title 21 of the Code of Federal Regulations, Part 50.

To obtain comparative results, the formulations were tested in pairs. In a first paired comparison, the cyclicsilicone with 3 wt % calcium silicate (Test Article A) was compared to the cyclicsilicone with 0.3 wt % triclosan (Test Article C), which is representative of the prior art. In a second paired comparison, the cyclicsilicone with 6 wt % calcium silicate (Test Article B) was compared to Test Article C. For each subject, a test article (A or B) was randomly assigned to one axilla and the opposite axilla received a control test article (C). Approximately 20 subjects completed each pair. See table XIII above for the exact compositions of the Test Articles.

Approximately 40 male and female subjects were selected to satisfy the following inclusion criteria consisting of age: 18 years or older and use of only a non-antimicrobial, fragrance-free soap product for all personal bathing for a minimum of 7 days prior. Subjects were free from axillary irritation, active psoriasis, eczema, skin cancer or dermatological conditions. Accepted subjects participated in a supervised wash using the above mentioned soap according to the following procedure. The right axilla was washed for approximately 10 seconds using a disposable towel saturated with a 2% solution of the standard soap. A fresh disposable towel was wetted under running water and used to rinse the axilla until all soap was removed. The axilla was gently patted dry using a dry disposable towel. The procedure was repeated for left axilla.

Subjects were given a treatment assignment number corresponding to the randomization following enrollment. Each subject received two-treatment application(s) at 0.40 g±0.02 g per axilla/application. The test article was applied by a technician who uniformly covered an approximate 4×6 cm area centered in the axillary vault. The amount of the test article used was determined by weighing each unit before and after each use. Subjects waited for a minimum of 10 minutes prior to receiving freshly laundered white T-shirts, which they wore during the test period.

The odor was evaluated for the subjects at 10 (±0.5) hours following the second treatment application. The odor evaluation scoring system was based on a range of underarm odor from no malodor (0), to moderate malodor (5), to extremely strong malodor (10). Odor measurements were made subjectively by trained judges familiar with the procedures and rating system.

The source data were the individual subject malodor scores assigned at the post-treatment evaluation by the three, trained judges. Separate analyses were conducted for each treatment pair (treatment article A verses C and treatment article B verses C). For the paired comparison evaluation of the test articles, the three judge average differences between treatments were analyzed using the distribution-free signed rank test as described in Non-parametric Statistical Methods, Hollander, M. and Wolfe, D. A., (1973) Chapter 3. The null hypothesis, which states that the difference between the paired test articles is equal to zero, was rejected if the signed rank test p-value is less than or equal to 0.05. The results are set forth in Tables XIV and XV, below.

TABLE XIV
Malodor Comparison of Paired Formulations

| | Cyclicsilicone | Cyclicsilicone |
|---|---|---|
| | 3% Calcium silicate | 0.3% Triclosan |
| | (Test Article A) | (Test Article C) |
| Mean Odor Score | 5.0 | 5.3 |
| % Improvement | 6.0 | — |
| Subjects, n | 21 | 21 |

TABLE XV
Malodor Comparison of Paired Formulations

| | Cyclicsilicone | Cyclicsilicone |
|---|---|---|
| | 6% Calcium silicate | 0.3% Triclosan |
| | (Test Article B) | (Test Article C) |
| Mean Odor Score | 4.9 | 5.5 |
| % Improvement | 11 | — |
| Subjects, n | 21 | 21 |

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A fluid personal care composition comprising calcium silicate and a vehicle, wherein the calcium silicate is capable of absorbing a malodorous compound.

2. The fluid personal care composition according to claim 1, wherein the composition further comprises a rheology modifier selected from the group consisting of solidifying agents, thickeners, and gellants.

3. The fluid personal care composition according to claim 1, wherein the calcium silicate is present in a concentration of from about 0.5 wt % to about 20 wt %.

4. The fluid personal care composition according to claim 1, wherein the calcium silicate has an oil absorption of between 200 ml/100 g and 600 ml/100 g, and a surface area (BET) of between 150 m²/g to 600 m²/g.

5. The fluid personal care composition according to claim 1, wherein the composition is a deodorant and is in a form selected from the group consisting of solid stick deodorants, liquid roll-on deodorants, aerosol, and pump spray deodorants, semi-solid gel deodorants, soap bars, and deodorant lotions and creams.

6. The fluid personal care composition according to claim 1, wherein the odor absorption capacity is greater than 200 μl/g.

7. The fluid personal care composition according to claim 1, wherein the vehicle is present in a concentration of from about 20 wt % to about 99 wt %.

8. The fluid personal care composition according to claim 1, wherein the vehicle is liquid at temperatures of 100° C. or less.

9. The fluid personal care composition according to claim 1, wherein the vehicle is selected from the group consisting of water, non-aqueous compounds that are liquid at temperatures of 100° C. or less, and mixtures thereof.

10. The fluid personal care composition according to claim 9, wherein the non-aqueous compound is selected from the group consisting of alcohols, polyalkoxylated glycols, volatile and nonvolatile liquid silicones, hydrocarbon and mineral oils, branched chain hydrocarbons, waxes and acrylic acid based polymers.

11. The fluid personal care composition according to claim 1, wherein the isovaleric acid absorption capacity is greater than 200 μl/g.

12. The fluid personal care composition according to claim 1, wherein the malodorous compound is selected from the group consisting of isovaleric acid, 2-pentenoic acid and 2-hexenoic acid.

13. A fluid personal care composition comprising:
(a) from about 0.5 wt. % to about 20 wt. % of calcium silicate; and
(b) from about 80 wt. % to about 99wt. % of a vehicle, which is liquid at temperatures below 100° C.;

14. A method of inhibiting body odor by applying to the skin an effective amount of a personal care composition comprising calcium silicate capable of absorbing volatilized malodors.
